(19) United States
(12) Patent Application Publication
(10) Pub. No.: US 2007/0276492 A1
(43) Pub. Date: Nov. 29, 2007

(54) Artificial spinal disc implant

(75) Inventors: Geoffrey Andrews, Cambridge (GB); Martin Cable, Cambridgeshire (GB); Robert Snell, Newmarket (GB); Scott Johnson, Newmarket (GB); Sinan Kiamil, Luton (GB); William Ellerkamp, London (GB)

Correspondence Address: WOLF GREENFIELD & SACKS, P.C., 600 ATLANTIC AVENUE, BOSTON, MA, 02210-2206, US

(73) Assignee: Ranier Limited, Cambridge (GB)

(21) Appl. No.: 11/431,121

(22) Filed: May 9, 2006

(51) Int. Cl. A61F 2/44

(52) U.S. Cl. 623/17.11

(57) ABSTRACT

An artificial spinal disc that may be implanted between adjacent vertebrae in the spine to replace, repair or augment a natural spinal disc. The spinal disc implant is characterized by one or more biomechanical properties that approximate those of a natural spinal disc and is durable enough to function in the body for long time periods, amongst other advantages.

### FIELD OF INVENTION

The present invention generally relates to artificial spinal disc implants.

### BACKGROUND OF INVENTION

A spinal disc lies between adjacent vertebrae in the spine. The disc stabilizes the spine and assists in distributing forces between vertebral bodies. A spinal disc includes an outer annulus fibrosis which surrounds an inner nucleus pulposus. The annulus fibrosis is a concentrically laminated structure of aligned collagen fibers and fibro cartilage which provides stability to resist torsional and bending forces. The nucleus pulposus comprises a gelatinous material which can absorb stresses acting on the disc.

A spinal disc may be displaced or damaged due to trauma, disease or other degenerative processes that can occur over time. For example, the annulus fibrosis may weaken and/or begin to tear which can result in the protrusion of the nucleus pulposus into a region of the spine (e.g., the vertebratal foramen) that includes spinal nerves. The protruding nucleus pulposus may press against spinal nerves causing pain, numbness, tingling, diminished strength and/or a loss of motion. Another common degenerative process is the loss of fluid from the nucleus pulposus. Such fluid loss can limit the ability of the nucleus pulposus to absorb stress and may reduce its height which can lead to further instability of the spine, as well as decreasing mobility and causing pain.

To address the conditions described above, a displaced or damaged spinal disc may be surgically removed from the spine and the two adjacent vertebrae may be fused together. Though this technique may initially alleviate pain and can improve joint stability, it also can result in the loss of movement of the fused vertebral joint.

Another solution has been to replace a damaged spinal disc with an artificial spinal disc implant. However, in general, such implants have been limited in their ability to adequately mimic the biomechanics of a normal healthy human spinal disc. For example, certain conventional artificial discs have a metallic bearing surface which may be hard and relatively non-deformable. When such discs are implanted, the metallic surface is in contact with relatively soft, cancellous bone. The difference in hardness between the metallic disc surface and the bone surface changes the distribution of stress in the spine compared to the stress distribution in a spine including natural discs. This stress re-distribution is known as stress-shielding and can expose regions of the spine adjacent the implant to increased mechanical stresses, enhancing the risk of further degeneration.

Other conventional spinal disc implants may have other limitations. For example, discs formed of a single material generally do not satisfactorily mimic the different properties of the nucleus pulposus and annulus fibrosis in a normal human spinal disc. To better approximate such performance, some discs have been developed that include a core of a first material contained within a shell of a second material. Such discs may be limited by progressive failure along the interface between the core and the shell over time, because of an abrupt change in properties (e.g., Young's modulus) at the interface.

An artificial spinal disc implant with certain biomechanical properties that better approximate those of a natural spinal disc and that is durable enough to function in the body for long time periods would be desirable.

### SUMMARY OF INVENTION

In one aspect, an artificial spinal disc implant includes a body and a first end plate provided with the body. The first end plate includes an outer surface formed of a material having a hardness of between 50 Shore D and 100 Shore D. The artificial spinal disc implant is constructed and arranged to replace, repair or augment a spinal disc separating adjacent vertebrae in a living being.

In another aspect, an artificial spinal disc implant includes a body having a nucleus region and an annulus region surrounding, at least in part, the nucleus region. A Young's modulus is varied across a portion of the annulus region. The portion has a volume between 20% and 60% of the volume of the body. The artificial spinal disc implant is constructed and arranged to replace, repair or augment a spinal disc separating adjacent vertebrae in a living being.

In another aspect, an artificial spinal disc implant includes a body and a first end plate provided with the body. The artificial spinal disc implant is constructed and arranged to replace, repair or augment a spinal disc separating adjacent vertebrae in a living being. The spinal disc implant has a bending stiffness between 0.5 Nm/degree and 5.0 Nm/degree.

In another aspect, an artificial spinal disc implant comprises a body including an upper outer surface, a lower outer surface and a sidewall between the upper outer surface and the lower outer surface. The sidewall defines a concave portion. The body includes an annulus region completely surrounding a nucleus region such that the annulus region separates the nucleus region from the upper surface, the lower surface, and the sidewall. At least a portion of the annulus region has a Young's modulus that is varied across the portion. The implant includes a first end plate formed integrally on the upper outer surface of the body such that there is no distinct interface between the first end plate and the body. The first end plate includes an outer surface formed of a material having a hardness of between 50 Shore D and 100 Shore D. The implant includes a second end plate formed integrally on the lower outer surface of the body such that there is no distinct interface between the second end plate and the body. The second end plate includes an outer surface formed of a material having a hardness of between 50 Shore D and 100 Shore D. The body, the first end plate and the second end plate comprise polyurethane material. The artificial spinal disc implant is constructed and arranged to replace, repair or augment a spinal disc separating adjacent vertebrae in a living being.

Other aspects, embodiments and features of the invention will become apparent from the following detailed description of the invention when considered in conjunction with the accompanying drawings. The accompanying figures are schematic and are not intended to be drawn to scale. In the figures, each identical, or substantially similar component that is illustrated in various figures is represented by a single numeral or notation.

For purposes of clarity, not every component is labeled in every figure. Nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions (if any), will control.

### BRIEF DESCRIPTION OF THE DRAWINGS

FIG. 1A shows a spinal disc implant in accordance with embodiments of the invention.

FIG. 1B shows a side view of the spinal disc implant of FIG. 1A.

FIG. 1C is a top view of the spinal disc implant of FIG. 1A.

FIG. 2A is a cross-section of a spinal disc implant in accordance with embodiments of the invention.

FIG. 2B is a schematic of the variation in modulus in the disc of FIG. 2A.

FIG. 3 shows a processing system suitable for manufacturing spinal disc implants in accordance with embodiments of the invention.

FIG. 4 is a graph of simulated bending moment data for spinal disc implants described in Example 1.

### DETAILED DESCRIPTION

An artificial spinal disc implant is provided that may be implanted between adjacent vertebrae in the spine to replace, repair or augment a natural spinal disc. As described further below, the spinal disc implant is characterized by one or more biomechanical properties that approximate those of a natural spinal disc and is durable enough to function in the body for long time periods, amongst other advantages.

The disc may include one or more end plates. The end plates may have an outer surface formed of a material having one or more properties (e.g., hardness) selected to complement those of the cancellous bone adjacent to end plate surfaces when the disc is implanted. Such a construction limits stress shielding and the resulting degenerative conditions in regions of the spine around the implant.

A body of the disc may include a nucleus region surrounded by an annulus region, similar to the structure of a natural spinal disc. The nucleus region may have a lower stiffness (e.g., characterized by Young's modulus) than that of the annulus region. In some embodiments, the stiffness may be graded across at least a portion of the body; for example, and without limitation, from a lower stiffness in the nucleus region to a higher stiffness in the annulus region. As described further below, this grading may lead to formation of a single unitary body without any distinct interfaces that would be formed at the juncture of two separate material layers. The absence of distinct interfaces can enhance durability of the disc since such interfaces can be sites of de-lamination during use which can lead to failure of the implant. Also, the graded portion may improve distribution of biomechanical loads and stresses which can also increase durability and/or limit degenerative conditions.

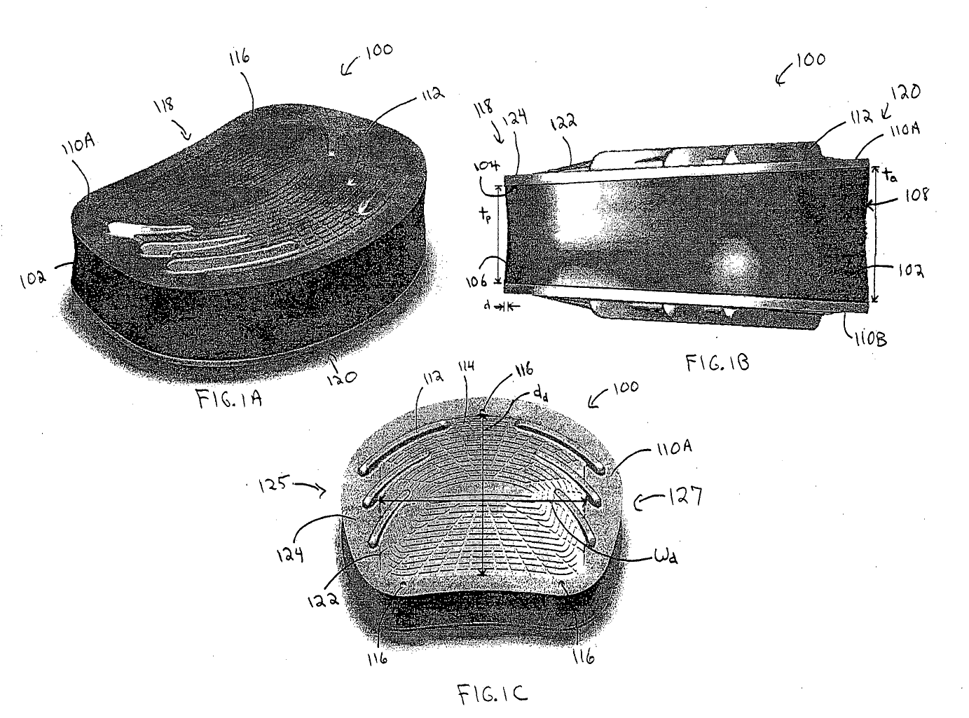

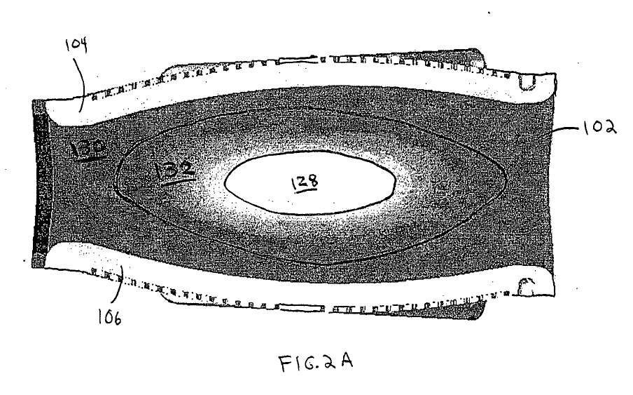

FIGS. 1A-1C show an artificial spinal disc implant 100 in accordance with embodiments of the invention. Disc 100 includes a body 102 having an upper surface 104, a lower surface 106 and a sidewall 108. The disc includes a first end plate 110A and a second end plate 110B which, in these illustrative embodiments, are on upper surface 104 and lower surface 106, respectively. It should be understood, however, that the end plates may be provided on the disc in other manners. The illustrated end plates include rib elements 112 which can fixate the disc to adjacent vertebrae when implanted, and a series of surface features 114 designed to enhance bone growth on the end plate and integration of the implanted disc within the body. The disc may also include one or more radiopaque markers 116 which can be used to identify disc position to ensure proper placement.

It should be understood that the spinal disc shown in FIGS. 1A-1C is an illustrative embodiment of the invention and that the invention is not limited to this or other embodiments shown in the figures and/or described in the Detailed Description.

Body 102 may be formed of any suitable material including biocompatible polymeric materials such as polyurethane materials. It should be understood that polyurethane materials include any polymeric material having a polyurethane component. Such materials may also include other polymeric components such as polycarbonate (e.g., polyurethane polycarbonate materials). Linear and cross-linked polyurethane materials may be suitable. In some embodiments, body 102 may be formed of a single type of polymeric material (e.g., a polyurethane material), as described further below. In embodiments in which the body is formed of only polyurethane material, different portions of the body may comprise polyurethane material having different stoichiometries and/or molecular weights.

In general, the dimensions of body 102 are selected to be suitable for implantation to replace, repair and/or augment a natural spinal disc. For example, the body may have a width of between about 37 to 47 (e.g., 42 mm); a depth between a posterior side 118 and an anterior side 120 of between about 27 to 37 mm (e.g., 32 mm); and, a thickness of between about 7 mm and 15 mm. In the illustrated embodiments, upper surface 104 and lower surface 106 may be angled so that posterior side 118 has a thickness tₚ that is less than a thickness tₐ of anterior side 120. For example, the angle may be between 6° and 12°. Such a construction may be advantageous for positioning the implant in the spine and/or for biomechanical performance once implanted.

As shown in FIG. 1B, sidewall 108 may be tapered toward the center of the body defining a concave surface that forms a curved “waist”. The “waist” construction may enhance flexibility of the disc and/or may reduce internal stresses, particularly in response to bending forces. In some cases, stress reduction may be enhanced when the concave portion extends inward a maximum distance (d) (e.g., perpendicular distance from the perpendicular line connecting the outermost edge of the upper and lower end plates) of between 0.5 mm and 5 mm; or, in some embodiments between 0.5 mm and 3 mm. It should be understood that the body may also include straight sidewalls and, thus, does include not a “waist” in all embodiments. Although the curved “waist” illustrated in the figures extends completely around the body, it is also contemplated that a curved waist may be provided at one or more regions around the body.

End plates 110A and 110B may be provided with the body in any suitable manner and using any suitable technique. For example, end plates 110A and 110B may be attached to a portion of the body. In some embodiments, the end plates are attached to the body during the manufacturing process as described further below. In these embodiments, the end plates and the body may form an integral (i.e., unitary) piece. That is, there are no distinct interfaces between respective end plates and the body. In these embodiments, the end plates may be attached without the use of a separate adhesive or glue; rather, the end plate material may be chemically bonded directly to the material of the body. For example, when the end plates are formed of polymeric material and the body is formed of polymeric material, chemical (e.g., covalent) bonds may be formed between the polymeric material of the end plate and polymeric material of the body. In some cases, polymeric chains may extend between the polymeric material of the end plate and polymeric material of the body. Eliminating the presence of distinct interfaces between the end plates and the body can enhance durability of the disc since such interfaces can be sites of de-lamination which can lead to failure of the disc.

It should also be understood that some embodiments may involve attaching the end plates to the body using a separate adhesive or glue. In such embodiments, a layer of adhesive or glue, thus, may be formed at the interface between the end plate and the body.

In general, the end plates may be formed of any suitable material including rigid polymeric materials such as certain polyurethane materials (e.g., polyurethane polycarbonate materials). As noted above, in some embodiments, outer surfaces of the end plates are formed of a material having properties selected to complement or match that of the cancellous bone adjacent end plate surfaces when the disc is implanted. The end plate material (e.g., polyurethane material) may be appropriately formulated to provide such desirable properties. For example, the end plate material may have a hardness or compressive modulus similar to that of cancellous bone and less than those of certain conventional metal end plate materials (e.g., titanium, cobalt-chrome alloys). For example, the material may have a hardness of between 50 Shore D and 100 Shore D, or between 70 Shore D and 90 Shore D. Shore hardness may be measured using procedures and instruments known to those of ordinary skill in the art. For example, suitable techniques for measuring Shore hardness for polymeric material are described in ASTM D2240. End plates having outer surfaces formed of materials having such hardness values can lead to minimal stress shielding and does not enhance degenerative conditions in regions of the spine around the implant.

In some embodiments, the end plates are formed entirely from a material having the above-noted hardness values. In these embodiments, the end plates may have a unitary construction. The end plates may also be formed of more than one material with the outer surface of the end plates being formed of a material having the above-noted hardness values and other portions of the end plates being formed of one or more other materials (including materials which may not have the above-noted hardness values).

It should be understood that not all embodiments include end plates having outer surfaces formed of materials having the above-noted hardness ranges. Also, in other embodiments, one of the end plates may have an outer surface formed of a material having the above-noted hardness values while the other end plate may not.

End plates 110A and 110B may have a dome-shape outer surface. As shown, end plates 110A and 110B have a dome-shaped region 122 that extends vertically from a flat portion 124. Though, it should be understood, that in other embodiments the entire outer surface of the end plate may be dome-shaped. The dome-shaped region may have dimensions selected to be compatible with the morphology of vertebral bodies. The domed-shape region may also facilitate the implant procedure. For example, the dome-shaped region may have a maximum dome height of between 0.75 mm and 3.0 mm, or between 0.75 mm and 1.5 mm. The dome-shaped region may also be characterized by having a width (w_{d}) and a depth (d_{d}). The width-to-depth ratio, for example, may be between 1.1 and 1.8 (e.g., 1.2).

End plates 110A and 110B are shown as including a series of rib elements 112. The rib elements can be used to fixate the disc in a proper position when implanted. For example, the rib elements may interact with the vertebrae to fixate the disc. In some embodiments, the rib elements fit into corresponding grooves that may be formed in the vertebrae. When positioned in the grooves, the rib elements are constructed and arranged to resist shear and rotation. It should be understood that the vertebrae may be otherwise prepared to accommodate the rib elements including, for example, roughening vertebrae surfaces.

In the illustrated embodiment, three rib elements are disposed on a left side 125 and a right half of the disc 127. As shown, the rib elements extend along curved paths with the rib element closest to anterior side 120 having the longest length and the rib element closest to the posterior side having the shortest length. The rib elements may have rounded edges, as shown, to facilitate fixation. In some embodiments, it may be preferable that the ribs have a height that does not exceed the height of dome-shaped region 122. Such a construction can facilitate proper placement of the disc within the vertebrae. For example, the ribs may have a height of less than 1.5 mm.

It should be understood that other types of fixation elements and/or fixation element arrangements are also possible.

End plates 110A and 110B also can include surface features 114 which are designed to enhance bone growth on the end plates and integration of the implant disc within the human body. In the illustrative embodiment, the surface features include macro-texture features (e.g., protrusions) that form a series of inter-connected channels defined in the outer surface of the end plates. The channels, for example, may have a width of between 100 microns and 750 microns (e.g., 400 microns). The macro-texture features may be protrusions having a width of between 200 microns and 400 microns (e.g., 300 microns) and a height of between 100 microns and 300 microns (e.g., 200 microns).

End plates 110A and 110B may also have micro-texture features (not shown). For example, the micro-texture features may have an average surface roughness (Rₐ) of between 0.1 micron and 10 micron. Average surface roughness (Rₐ) may be measured using procedures and instruments known to those of ordinary skill in the art including surface profilometers. The micro-texture features may also enhance bone growth on the end plates. The features may be configured to encourage/facilitate osteointegration which may make the end plate surface osteoconductive even if the end plate material may not be known as an osteoconductive material.

Outer surfaces of end plates 110A and 110B may also be coated with a suitable material to enhance bone growth. For example, suitable coating materials include osteoconductive materials (e.g., osteoconductive ceramics or osteoconductive polymers), osteophylic materials and bioactive coatings (e.g., bone morphogenic proteins, BMP).

As noted above, the disc may include one or more radiopaque markers 116 which can be used to identify disc position and to ensure proper placement. In general, radiopaque markers 116 may be formed of any suitable material including those that are visible with x-rays systems and are compatible with the body. Suitable materials for the markers include certain metals (such as titanium, tantalum, gold, tungsten, platinum and mixtures thereof) and polymeric materials loaded with a radio-opacifier (e.g., barium compounds including barium sulfate). The radiopaque markers, for example, may be circular regions. The circular regions may have a diameter of less than 2 mm.

In the illustrative embodiments, three radiopaque markers 116 are arranged to define corners of a triangular shape. As shown, two of the markers are positioned on posterior side 118 of the disc and one on anterior side 120. Markers 116 may be positioned on flat portion 124 along the periphery of the outer surface of end plate 110A. Such an arrangement enables precise location and orientation of the disc which can ensure proper placement of the implant. Other types and arrangements of radiopaque markers are also possible.

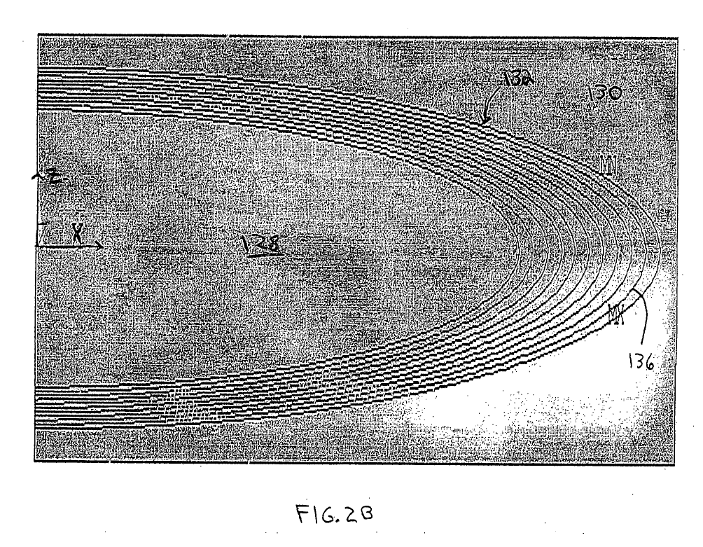

FIGS. 2A and 2B illustrate an embodiment in which body 102 includes a nucleus region 128 completely surrounded, so as to be encapsulated, by an annulus region 130. Thus, the nucleus region is separated from upper surface 104, lower surface 106 and sidewall 108 of the spinal disc and does not contact the end plates. In certain embodiments, the annulus region may surround the nucleus region to a lesser extent. For example, the annulus region may surround the majority of, but not the entire, surface area of the nucleus region (e.g., between 50% and 90% of the surface area of the nucleus region). For example, part of the top and/or bottom of the nucleus may be covered by the annulus, while certain region or regions of the top and/or bottom of the nucleus are not surrounded by the annulus. Similarly, some, but not, all of the side of the nucleus may be covered by the annulus, while certain region or regions of the side of the nucleus are not surrounded by the annulus.

Nucleus region 128 may have different properties than annulus region 130. The nucleus region may have properties selected to mimic the function of the nucleus pulposus in a natural spinal disc; while, the annulus region may have properties selected to mimic the function of the annulus fibrosis in a natural spinal disc. For example, the nucleus region may be relatively soft and compliant; while, the annulus region may be stiffer and stronger. In particular, the nucleus region may have a Young's modulus that is lower than a Young's modulus within the annulus region.

Young's modulus is a measure of the stiffness of a material and may be measured according to conventional techniques. For example, a nanoindentation technique may be used to measure Young's Modulus at different locations within the body 102. The disc may be prepared for nanoindentation testing by taking a suitable cross-section through the body to form a desired sample surface (e.g., a surface that exposes the nucleus region and the annulus region). Nanoindentation involves applying a known force to a location on the sample surface with an indenter and measuring the resulting depth of penetration into the material. The Young's modulus (as well as other properties) may be calculated from the relationship between the applied force and the resulting depth. The test can be repeated at different locations (e.g., locations within the nucleus region, locations within the annulus region) to determine the variation of Young's modulus across the body.

As noted above, in some embodiments, the annulus region may include a graded portion 132 across which a property, such as Young's modulus, is varied. That is, the Young's modulus changes with distance in a direction across the portion. As described further below, in some cases, it is preferred that the Young's modulus increase with distance away from the nucleus region. The graded portion 132 may enhance the ability of the disc to absorb and effectively distribute biomechanical loads and stresses. The graded portion can also enable elimination of a distinct interface between the nucleus region and the annulus region, as described further below.

It should be understood that though the description herein focuses on the Young's modulus, other properties may also be graded such as compressive modulus, tensile strength, and/or hardness in similar manners. Material composition characteristics (e.g., polymeric material stoichiometry) may also be graded in a similar manner which can lead to the grade in properties, as described further below. Known techniques (e.g., FTIR spectrophotometry) may be used to map the change in material composition.

FIG. 2B depicts an example of an annulus region including a graded portion having a Young's modulus that varies. As shown, each contour line 136 represents a different Young's modulus value. In embodiments in which the Young's modulus is increased across portion 132 with distance away from the nucleus region, it should be understood that respective contour lines represent higher Young's modulus values as distance from the nucleus is increased. Thus, the outermost contour line (i.e., furthest from the nucleus region) represents the highest Young's modulus value and the innermost contour line (i.e., closest to the nucleus region) represents the lowest Young's modulus value.

As shown, the Young's modulus may be graded differently along different directions within the portion. For example, the Young's modulus may be graded differently along each of the x-axis, y-axis, and z-axis. Thus, body 12 may be anisotropic with regard to Young's modulus. The change in modulus (i.e., grade) is greater in regions in which contour lines 136 lie closer together.

In some embodiments, the Young's modulus may be graded continuously with distance across the portion. The continuous grade may be substantially linear, or may be non-linear (e.g., parabolic). In other embodiments, the grade may be discontinuous. For example, the discontinuous grade may be step-wise.

The Young's modulus may be graded in any suitable manner and the specific grade depends on the desired disc properties. In certain embodiments, it may be preferable for the Young's modulus to increase with distance away from the nucleus region as noted above. However, the spinal discs of the invention are not limited to this design.

In some embodiments, the entire annulus region 130 has a graded Young's modulus. Thus, in these embodiments, portion 132 extends across the entire region 130. In other embodiments, the Young's modulus may be graded across only a portion of the annulus region. Thus, in these embodiments, portion 132 does not extend across the entire region 132. In these embodiments, portion 132 may separate the nucleus region from a portion of the annulus region which includes constant properties (e.g., Young's modulus). As shown in FIG. 2B, the portion of the annulus region lying surrounding graded portion 132 has a constant Young's modulus.

The relative volume of the graded portion(s) 132 compared to the rest of the body can contribute to the overall properties of the disc. For example, in some embodiments, it is preferred that the portion have a volume between 10% and 90% of the volume of the material body. In some cases, it is preferable that the volume of the portion be between 20% and 60% of the volume of the body; and in some cases between 35% and 45% (e.g., 40%). Portions having volume between 20% and 60%, and especially between 35% and 45%, may be particularly well-suited for providing a balance properties that mimic the biomechanics of a natural spinal disc.

As noted above, body 102 may be formed of a single type of polymeric material (e.g., a polyurethane material). In embodiments that include a nucleus region and an annulus region, both the nucleus region and the annulus region (including any graded portion that may be present) may be formed of the same type of polymeric material. For example, the nucleus region may be formed of a polyurethane material having a first stoichiometry, while the annulus region may be formed of the same polymeric material composition having a second stoichiometry different than the first stoichiometry. The material in the nucleus region may have a lower molecular weight than the material in the annulus region which results in the difference in stoichiometry. The difference in stoichiometry can also lead to the difference in properties (e.g., Young's modulus) between the nucleus and the annulus region. In embodiments including a portion having graded properties, the stoichiometry of the polymeric material may be similarly graded and can lead to the grade in properties.

In embodiments in which the nucleus region and the annulus region (including graded portion) are formed of the same material, no distinct interfaces are formed between the two regions. As noted above, the absence of distinct interfaces can enhance durability of the disc since such interfaces can be sites of de-lamination during use which can lead to failure of the implant. When the nucleus region and the annulus region are formed of the same material, chemical (e.g., covalent) bonds may be formed between the polymeric material of the nucleus region and the polymeric material of the annulus region. In some cases, polymeric chains may extend between the polymeric material of the nucleus region and the polymeric material of the annulus region.

In one preferred embodiment, spinal disc 100 has a width of between about 37 to 47 (e.g., 42 mm); a depth between posterior side 118 and anterior side 120 of between about 27 to 37 mm (e.g., 32 mm); and, a thickness at posterior side 118 of between about 9 mm and 12 mm. In this embodiment, the thickness at the anterior side is greater than the thickness at the posterior side. For example, the angle defined by the surface extending from the posterior side to the anterior side is between 6° and 12° (e.g., 6°, 9°, 12°). In this embodiment, the body includes a waist defined by a concave portion of the sidewall of the body. The concave portion, for example, extends inward a maximum distance (d) of between 0.5 mm and 5 mm, or 0.5 mm and 3 mm.

In this embodiment, the body of the spinal disc includes a nucleus region and an annulus region completely surrounding the nucleus region. The nucleus region has a lower Young's modulus than that of the annulus region with a Young's modulus that is increased across a graded portion of the annulus region. The graded portion, for example, has a volume of between 20% and 60 of the total volume of the body.

In this embodiment, the entire spinal disc 100 (including body and end plates) may be formed of a polyurethane material (e.g., polyurethane polycarbonate materials). Chemical (e.g., covalent) bonds may be formed between polyurethane material of the nucleus region and polyurethane material of the annulus region and polyurethane material of the end plates. In some cases, polymeric chains may extend between polyurethane material in the nucleus region and polyurethane material in the annulus region and polyurethane material in the end plates.

When the entire disc is formed of a single material (e.g., a polyurethane material), there may be no distinct interfaces (e.g., interfaces formed between two separate materials) formed within the entire disc. As noted above, the absence of distinct interfaces can enhance durability of the disc since such interfaces can be sites of de-lamination during use which can lead to failure of the implant. Also, when formed entirely of polymeric materials (e.g., polyurethane materials), the disc may be MRI compatible which is advantageous once implanted.

In this preferred embodiment, the end plate material may have a hardness or compressive modulus similar to that of cancellous bone. For example, the material may have a hardness of between 50 Shore D and 100 Shore D. As described above, such hardness values can lead to minimal stress shielding and does not enhance degenerative conditions in regions of the spine around the implant.

It should be understood that the preferred embodiment described in the five preceding paragraphs is not to be considered limiting. Other embodiments of the invention may include some, but not all, of the features described in connection with this embodiment. Also, it should be understood that other embodiments of the invention may include any combination of the features described throughout the Detailed Description.

Spinal disc implants can be designed, as described above, to have properties that are similar to that of a natural spinal disc.

The disc may have an axial stiffness in the range between 1000 N/mm and 3500 N/mm. Axial stiffness is expressed as a force per unit displacement for an applied compressive force which acts perpendicular to the disc mid-plane.

The disc may have a torsional stiffness of between 0.5 Nm/degree and 10 Nm/degree. The torsional stiffness of the disc is described as the force per unit displacement and refers to an isolated implant. Torsional stiffness is calculated by applying a torque using appropriate testing apparatus, through the central loading axis of the implant and recording the angular displacement. The stiffness is subsequently calculated by dividing the applied torque by the angular displacement.

The disc may have a flexural (i.e., bending) stiffness of between 0.5 Nm/degree and 5.0 Nm/degree, between 1.0 Nm/degree and 4.0 Nm/degree, or between 1.0 Nm/degree and 3.0 Nm/degree, for flexion, extension and lateral bending motions (also any motions in between these). The flexural stiffness of the disc is described as the force per unit displacement and, in this case, refers to an isolated implant. One suitable method for measuring flexural stiffness involves the application of a load which is displaced with respect to the central loading axis (i.e., the position at which a point load results in compression only and does not induce any change in angular displacement) of the implant to induce an angular deflection. The moment arm is given by the distance between the central loading axis and loading application point. A goniometer or suitable image capture system may be used to provide real time display of the change in angular displacement with applied load. The bending moment is calculated using simple trigonometry applied to the moment arm and angular displacement. The bending stiffness is subsequently calculated by dividing the applied torque by the angular displacement. It should be noted that the method of calculating the bending stiffness does not take into account the precise location of the center of rotation of the device and also assumes that the center of rotation does not change throughout the load application cycle.

In general, any suitable process may be used to manufacture spinal disc implants of the invention. Suitable processes have been described generally in commonly-owned U.S. patent application Ser. No. 10/530,919, filed Apr. 8, 2005, which is incorporated herein by reference and is based on International Application No. PCT/GB2003/004352, filed Oct. 8, 2003 which published as International Publication No. 2004/033516. Though one suitable process is further described below, it should be understood that other processes may also be suitable.

The process can involve creating a mixture of reagents including a multifunctional isocyanate, a polyol and, optionally, a chain extender. Any suitable multifunctional isocyanate (e.g., diisocyanate), polyol (e.g., a hydroxy-terminated ester, ether or carbonate diol) and chain extender may be used.

The reagents may be mixed using conventional techniques. In some embodiments, the reagents are mixed vigorously so that the reagents are mixed on a molecular level, though the process is not limited to such mixing. The two or more reagents may be mixed via a reactive injection processing technique (e.g., conventional RIM or SRIM processes). In one embodiment, the process uses an impingement mixing head. It may be preferred that the reagents are mixed quickly so that the resultant mixture is substantially homogeneous nearly immediately after mixing (e.g., within several seconds) after mixing.

When forming a spinal disc having regions of different polyurethane compositions (e.g., nucleus region, annulus region, graded portion), the relative amounts of reagents (e.g., multifunctional isocyanate and/or polyol) being mixed may be changed at selected time(s) during the process. This changes the relative reagent concentration within the mixture so that the mixture includes a portion of first relative reagent concentration and a portion of second relative reagent concentration. It should be understood that the relative amounts of the reagents being mixed may be changed any number of times to produce additional portions having different relative reagent concentrations. As described further below, further processing of a mixture including portions having different relative reagent concentrations can form polyurethane compositions having different characteristics (e.g., stoichiometry and/or molecular weight) which enables formation of a disc including regions having different properties (e.g., nucleus region, annulus region, and graded portion), as described further below.

The resulting mixture is further processed by controlling temperature conditions to dictate progress of the polymerization reactions. For example, the mixture may be introduced into an extruder including a heated barrel. As the mixture is conveyed in a downstream direction within the barrel, the reagents react to form polyurethane material compositions. As described above, portions of the mixture having different relative reagent concentrations may react within the extruder to form polyurethane compositions having different characteristics (e.g., stoichiometry and/or molecular weight). The temperature of the barrel may be controlled to provide desired temperature conditions for the reactions. It should be understood that typically polymerization reactions between the reagents continue after extrusion, as described further below. Additional reagents (e.g., chain extenders) can be introduced into the mixture within the extruder and can participate in the polymerization reaction.

The process conditions and/or physical and chemical properties of the mixture may be monitored during extrusion using instruments such as sensors (e.g., temperature and pressure), rheometers, densitometers, spectrophotometers or any combination thereof.

The process further involves processing the mixture (e.g., which may include polymerized polyurethane material in addition to the reagents) into a suitable shape. For example, after extrusion, the mixture may be injected into a mold cavity and solidified to form the desired shape. As noted above, the mixture being processed may include portions having different polyurethane material compositions. These portions can lead to formation of a body including different regions having different material properties and compositions (e.g., nucleus region, annulus region and graded portion). The different portions may be injected into the mold simultaneously or in consecutive steps. As noted above, polymerization reactions may continue within the mold so that polyurethane material of different portions may be chemically bonded with one another to form an integral structure without distinct interfaces as described above.

Different components of the spinal disc may be formed in different molding steps. For example, the body of the spinal disc may be formed in a first molding step, while the end plates may be formed in a second molding step. In such embodiments, the body may be formed in a first mold cavity and, prior to solidification of the body, the mold may be modified to form a second mold cavity in which the end plates are formed. Polymerization reactions may continue within the mold so that the polyurethane material of the end plates are chemically bonded with polyurethane material from the body to form a structure without distinct interfaces, as described above.

After the shaping process, the structure may be recovered and may undergo any final processing steps (e.g., applying coatings to end plates surfaces) desired to form the final disc.

It should be understood that the above-described process may include a number of variations. Other processing techniques may also be suitable.

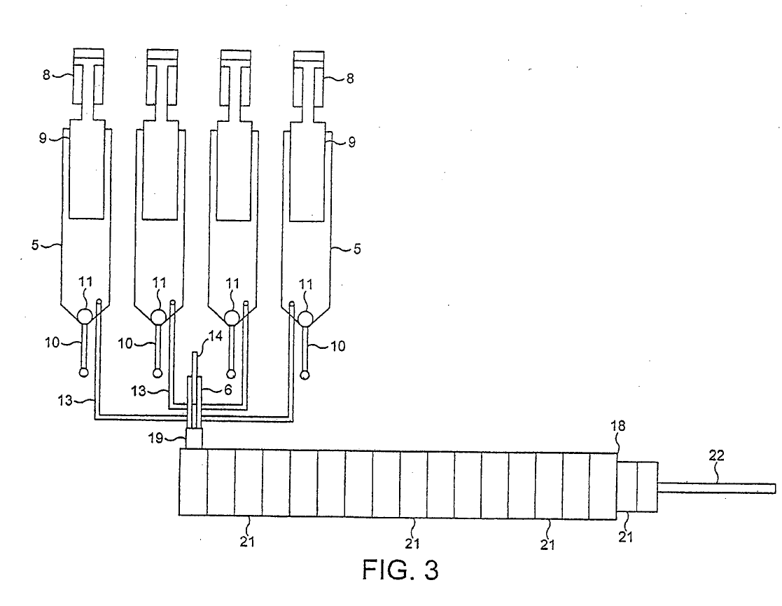

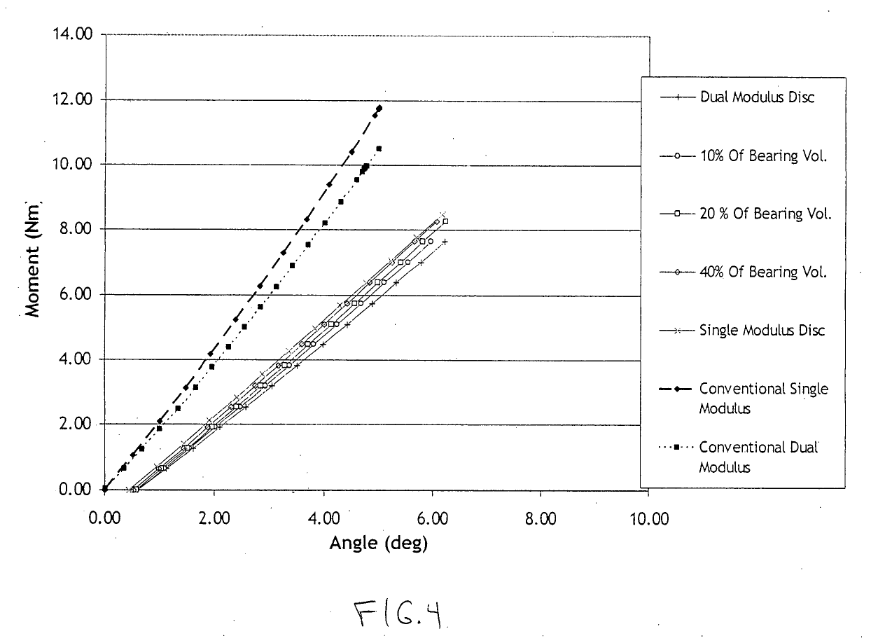

FIG. 3 illustrates a polymer processing system 30 which may be used in connection with the process described above to manufacture spinal discs (or components thereof) of the invention. System 10 includes a series of injection lances 5 which contain reagents (e.g., multifunctional isocyanate, polyol). For example, each lance may contain a separate reagent, or mixture of reagents. It should be understood that other systems may include additional lances. Reagents may be supplied to the lances through an inlet pipe 10 and a valve assembly 11. The injection lances include a hydraulic cylinder 8 which is designed to force a plunger 9 distally to inject reagents contained in the respective lances into outlet pipes 13. In the illustrative embodiment, the respective outlet pipes are connected to a mix head 6. The injection of reagents from the lances may be controlled so that the reagents are mixed within the mix head.

In this embodiment, an outlet of the mix-head is connected to an extruder 18. As shown, a rheometer 19 is positioned at the outlet of the mix-head to measure viscosity of the mixture. The extruder includes a series of heaters 21 arranged along the length of the barrel which may be operated to provide desired temperature conditions for the polymerization reactions as described above. The extruder includes a die 22 through which the mixture is extruded upon injection into a mold (not shown). It should be understood that the extruder may include a number of other components such as sensors (e.g., temperature and pressure), densitometers, and spectrophotometers, amongst others.

In some embodiments, the system may include a control system for controlling various process parameters including the rate at which reagents are delivered to the mix-head, the rate at which the resultant mixture is introduced into the extruder and temperature conditions of the extruder, amongst others, in response to inputs from the various measurement instruments.

It should be understood that the system described above may include a number of modifications and that other systems may also be suitable in the manufacture of spinal discs of the invention.

The following example is meant to be illustrative and is not intended to be limiting in any way.

### EXAMPLE 1

This example illustrates bending stiffness data for spinal disc implants in accordance with certain embodiments of the invention. The data was obtained from mathematical simulations based on a number of discs having a design similar to the disc illustrated in FIGS. 1A-1C and a number of discs having a conventional design. The discs similar to those illustrated in FIGS. 1A-1C included: (a) a dual modulus disc; (b) a single modulus disc; (c) a disc including a nucleus region, annulus region and graded portion having 10% volume of body; (d) a disc including a nucleus region, annulus region and graded portion having 20% volume of body; (e) a disc including a nucleus region, annulus region and graded portion having 40% volume of body. The discs having a conventional design included: (f) a single modulus disc having titanium end plates; (g) a dual modulus disc (with no graded portion) having titanium end plates.

The simulations were based on a disc positioned between an upper and a lower vertebrae. The upper and lower surfaces of the vertebrae were positioned parallel to one another at the onset of the simulation. A pre-load of 400 N was applied to the top of the upper vertebrae, then an off-set load of increasing magnitude was applied to the anterior surface of the upper vertebrae to induce bending, while the lower vertebrae remained static. The bending moment was determined from the angle between the vertebrae (which is calculated using simple trigonometry) and the magnitude of the applied off-set load.

FIG. 5 is a graph showing the bending moment versus the angle. The bending stiffness was calculated using the bending moment. The bending stiffness for each disc (following the notation above) was as follows: (a) 1.42 Nm/degree; (b) 1.52 Nm/degree; (c) 1.48 Nm/degree; (d) 1.51 Nm/degree; (e) 1.54 Nm/degree; (f) 11.78 Nm/degree; (g) 10.53 Nm/degree.

The example shows that the bending stiffnesses determined from the simulations of discs having a design similar to the disc illustrated in FIGS. 1A-1C are lower than those of discs having a conventional design.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. An artificial spinal disc implant for replacing, repairing and/or augmenting a spinal disc in a living being, comprising:
a body; and
a first end plate provided with the body, the first end plate including an outer surface formed of a material having a hardness of between 50 Shore D and 100 Shore D,
wherein the spinal disc implant is constructed and arranged for placement between adjacent vertebrae in a living being.

2. The implant of claim 1, wherein the body includes an upper outer surface, a lower outer surface and a sidewall between the upper outer surface and the lower outer surface, wherein the sidewall defines a concave portion.

3. The implant of claim 2, wherein the concave portion extends inward a maximum distance of between 0.5 mm and 5 mm.

4. The implant of claim 2, wherein the concave portion extends inward a maximum distance of between 0.5 mm and 3 mm.

5. The implant of claim 1, further comprising a second end plate provided with the body.

6. The implant of claim 5, wherein the second end plate includes an outer surface formed of a material having a hardness of between 50 Shore D and 100 Shore D.

7. The implant of claim 5, wherein the first end plate and the second end plate comprise polymeric material.

8. The implant of claim 7, wherein the polymeric first end plate and the polymeric second end plate comprise polyurethane material.

9. The implant of claim 8, wherein the polyurethane material is polyurethane polycarbonate.

10. The implant of claim 7, wherein the body comprises polymeric material and the polymeric material of the first end plate and the polymeric material of the second end plate are chemically bonded to the polymeric material of the body.

11. The implant of claim 7, wherein the first end plate and the second end plate are formed integral with the body.

12. The implant of claim 5, wherein the outer surface of the first end plate includes a series of rib-shaped members.

13. The implant of claim 1, wherein the outer surface of the first end plate includes a series of first surface features having a width of between 100 micron and 750 micron.

14. The implant of claim 1, wherein the outer surface of the first end plate includes a series of second surface features having an average surface roughness of between 0.1 micron and 10 micron.

15. The implant of claim 1, wherein the first end plate has a domed-shape region.

16. The implant of claim 15, wherein the dome-shaped region has a height between 0.75 mm and 3.0 mm.

17. The implant of claim 15, wherein the dome-shaped region has a height between 0.75 mm and 1.5 mm.

18. The implant of claim 15, wherein the dome-shaped region has a width-to-depth ratio of between 1.1 and 1.8.

19. The implant of claim 1, further comprising a plurality of radiopaque markers in the first end plate.

20. The implant of claim 19, wherein the radiopaque markers are arranged in a triangular pattern.

21. The implant of claim 19, wherein the radiopaque markers are circular regions having a diameter of less than 2 mm.

22. The implant of claim 1, wherein the implant is MRI compatible.

23. The implant of claim 1, wherein the body includes a nucleus region and an annulus region surrounding, at least in part, the nucleus region.

24. The implant of claim 23, wherein the nucleus region and the annulus region comprise polymeric material.

25. The implant of claim 24, wherein the nucleus region and the annulus region comprise polyurethane material.

26. The implant of claim 23, wherein the body includes an upper outer surface, a lower outer surface and a sidewall between the upper outer surface and the lower outer surface, the annulus region separating the nucleus region from the upper surface, the lower surface, and the sidewall.

27. The implant of claim 23, wherein a Young's modulus is varied across at least a portion of the annulus region.

28. The implant of claim 27, wherein the body has a volume and the portion having the Young's modulus that is varied has a volume between 20% and 60% of the volume of the body.

29. The implant of claim 27, wherein the Young's modulus in the portion is increased with increasing distance from the nucleus region.

30. The implant of claim 27, wherein the portion having the Young's modulus that is varied comprises polymeric material having a stoichiometry that is varied across the portion.

31. The implant of claim 1, wherein the first end plate includes an outer surface having a hardness of between 70 Shore D and 90 Shore D.

32. An artificial spinal disc implant for replacing, repairing and/or augmenting a spinal disc in a living being comprising:
a body having volume, the body comprising:
a nucleus region; and
an annulus region surrounding, at least in part, the nucleus region, wherein a Young's modulus is varied across a portion of the annulus region, the portion having a volume between 20% and 60% of the volume of the body.
wherein the spinal disc implant is constructed and arranged for placement between adjacent vertebrae in a living being.

33. The implant of claim 32, wherein the body includes an upper outer surface, a lower outer surface and a sidewall between the upper outer surface and the lower outer surface, wherein the sidewall defines a concave portion.

34. The implant of claim 33, wherein the concave portion extends inward a maximum distance of between 0.5 mm and 5 mm.

35. The implant of claim 32, further comprising a first end plate provided with the body and a second end plate provided with the body.

36. The implant of claim 35, wherein the first end plate and the second end plate include an outer surface formed of a material having a hardness of between 50 Shore D and 100 Shore D.

37. The implant of claim 35, wherein the first end plate and the second end plate comprise polymeric material.

38. The implant of claim 37, wherein the first end plate and the second end plate comprise polyurethane material.

39. The implant of claim 38, wherein the polyurethane material comprises polyurethane polycarbonate.

40. The implant of claim 37, wherein the body comprises polymeric material and the polymeric material of the first end plate and the polymeric material of the second end plate are chemically bonded to the polymeric material of the body.

41. The implant of claim 37, wherein the first end plate and the second end plate are formed integral with the body.

42. The implant of claim 37, wherein the first end plate and the second end plate include a series of rib-shaped members.

43. The implant of claim 37, wherein the first end plate and the second end plate have a domed-shape region.

44. The implant of claim 32, wherein the nucleus region and the annulus region comprise polymeric material.

45. The implant of claim 44, wherein the nucleus region and the annulus region comprise polyurethane material.

46. The implant of claim 32, wherein the body includes an upper outer surface, a lower outer surface and a sidewall between the upper outer surface and the lower outer surface, the annulus region separating the nucleus region from the upper surface, the lower surface, and the sidewall.

47. The implant of claim 32, wherein the Young's modulus in the portion is increased with increasing distance from the nucleus region.

48. The implant of claim 47, wherein the Young's modulus in the portion is increased continuously with increasing distance from the nucleus region.

49. The implant of claim 32, wherein the portion having the Young's modulus that is varied comprises polymeric material having a stoichiometry that is varied across the portion.

50. An artificial spinal disc implant for replacing, repairing and/or augmenting a spinal disc in a living being, comprising:
a body; and
a first end plate provided with the body,
wherein the spinal disc implant is constructed and arranged for placement between adjacent vertebrae in a living being and the spinal disc implant has a bending stiffness between 0.5 Nm/degree and 5.0 Nm/degree.

51. The implant of claim 50, wherein the bending stiffness is between 1.0 Nm/degree and 4.0 Nm/degree

52. The implant of claim 50, wherein the body includes an upper outer surface, a lower outer surface and a sidewall between the upper outer surface and the lower outer surface, wherein the sidewall defines a concave portion.

53. The implant of claim 50, further comprising a second end plate provided with the body.

54. The implant of claim 53, wherein the first end plate and the second end plate comprise polymeric material.

55. The implant of claim 54, wherein the first end plate and the second end plate comprise polyurethane material.

56. The implant of claim 54, wherein the polymeric material of the first end plate and the polymeric material of the second end plate are chemically bonded to polymeric material of the body.

57. The implant of claim 50, wherein the body comprises polymeric material.

58. The implant of claim 50, wherein the first end plate has an outer surface having a domed-shape region.

59. The implant of claim 50, wherein the body includes a nucleus region and an annulus region surrounding, at least in part, the nucleus region.

60. The implant of claim 59, wherein a Young's modulus is varied across at least a portion of the annulus region.

61. The implant of claim 60, wherein the body has a volume and the portion having the Young's modulus that is varied has a volume between 20% and 60% of the volume of the body.

62. The implant of claim 60, wherein the Young's modulus in the portion is increased with increasing distance from the nucleus region.

63. The implant of claim 50, wherein the first end plate includes an outer surface having a hardness of between 50 Shore D and 100 Shore D.

64. An artificial spinal disc implant for replacing, repairing and/or augmenting a spinal disc in a living being, comprising:
a body including an upper outer surface, a lower outer surface and a sidewall between the upper outer surface and the lower outer surface, the sidewall defining a concave portion,
the body including an annulus region completely surrounding a nucleus region such that the annulus region separates the nucleus region from the upper surface, the lower surface, and the sidewall,
at least a portion of the annulus region having a Young's modulus that is varied across the portion,
a first end plate formed integrally on the upper outer surface of the body such that there is no distinct interface between the first end plate and the body, the first end plate including an outer surface formed of a material having a hardness of between 50 Shore D and 100 Shore D,
a second end plate formed integrally on the lower outer surface of the body such that there is no distinct interface between the second end plate and the body, the second end plate including an outer surface formed of a material having a hardness of between 50 Shore D and 100 Shore D
the body, the first end plate and the second end plate comprising polyurethane material,
wherein the artificial spinal disc implant is constructed and arranged to replace, repair or augment a spinal disc separating adjacent vertebrae in a living being.

65. The implant of claim 64, wherein the Young's modulus in the portion is increased with increasing distance from the nucleus region.

66. The implant of claim 64, wherein there is no distinct interface between the annulus region and the nucleus region.

67. The implant of claim 64, wherein the spinal disc implant has a bending stiffness between 0.5 Nm/degree and 5.0 Nm/degree.
